(19) United States
(12) Patent Application Publication
(10) Pub. No.: US 2022/0082521 A1
(43) Pub. Date: Mar. 17, 2022

(54) Metal Panel Crack Detection Sensor and Metal Panel Crack Detection Device Including the Same

(71) Applicants: Hyundai Motor Company, Seoul (KR); Kia Corporation, Seoul (KR)

(72) Inventor: Hyuk Lee, Seoul (KR)

(21) Appl. No.: 17/371,395

(22) Filed: Jul. 9, 2021

(30) Foreign Application Priority Data
Sep. 16, 2020 (KR) 10-2020-0118923

(51) Int. Cl. G01N 27/20

(52) U.S. Cl. CPC G01N 27/20; G01M 17/00

(57) ABSTRACT

An embodiment metal panel crack detection sensor includes a cover filled with a conductive material, a measurement line configured to measure a voltage generated in the conductive material, a power line configured to supply power to the conductive material, a metal member, and a metal panel comprising an insulating coating layer formed on the metal member, wherein the cover is attached to the metal panel, and wherein when the metal panel has a crack, the conductive material is configured to move into the crack.

### CROSS-REFERENCE TO RELATED APPLICATIONS

This application claims the benefit of Korean Patent Application No. 10-2020-0118923, filed in the Korean Intellectual Property Office on Sep. 16, 2020, which application is hereby incorporated herein by reference.

### TECHNICAL FIELD

The present invention relates to a sensor.

### BACKGROUND

Metal panels are used in various fields such as vehicles, ships, and household electrical appliances. The metal panels are mostly produced at a high speed by automation systems, and the metal panels have very complicated shapes.

Because fine defects, such as cracks, generated during processes cause deterioration in the quality of products and losses of interests and images of companies, studies are being actively conducted on technologies for detecting defects of panel products in production lines.

Various methods are used to detect defects on surfaces of metal panels. Among the various methods, a method of detecting the presence or absence of the defect by allowing an operator to directly check the surface of the metal panel with the naked eye is widely used. However, there is a limitation in checking the surface of the metal panel directly by the operator with the naked eye. In a case in which the surface of the metal panel has a fine defect, it is difficult to check and detect all the defects.

The above information disclosed in this Background section is only for enhancement of understanding of the background of the invention, and therefore it may contain information that does not form the prior art that is already known to a person of ordinary skill in the art.

### SUMMARY

The present invention relates to a sensor. Particular embodiments relate to a metal panel crack detection sensor and a metal panel crack detection device including the same.

Embodiments of the present invention provide a metal panel crack detection sensor and a metal panel crack detection device including the same, which are capable of accurately detecting a crack in a metal panel.

An exemplary embodiment of the present invention provides a metal panel crack detection sensor including a cover filled with a conductive material, a measurement line configured to measure a voltage generated in the conductive material, and a power line configured to supply power to the conductive material, in which an insulation coating layer to which the cover is attached is formed on a metal member, and in which when the metal panel including the insulation coating layer has a crack, the conductive material moves into the crack.

The metal panel crack detection sensor may further include a bonding agent configured to attach the cover to the metal panel.

The power line may not be in contact with the measurement line.

An end of the measurement line may have a pointy shape so that the measurement line is inserted into the cover, and an end of the power line may have a pointy shape so that the power line is inserted into the cover.

The metal panel may include a vehicle metal panel.

The conductive material may include graphite powder.

Another exemplary embodiment of the present invention provides a metal panel crack detection device including a power supply configured to supply power to a power line of a metal panel crack detection sensor, a measuring device configured to measure a voltage generated in a conductive material of the metal panel crack detection sensor from a measurement line of the metal panel crack detection sensor, and a ground line connected to a negative terminal of the power supply and a negative terminal of the measuring device. The metal panel crack detection sensor includes a cover filled with the conductive material, the measurement line configured to measure a voltage drop occurring in the conductive material, and the power line configured to supply power to the conductive material, in which an insulation coating layer to which the cover is attached is formed on a metal member, in which when the metal panel including the insulation coating layer has a crack, the conductive material moves into the crack, and in which the measuring device measures the voltage drop occurring in the conductive material by using the measurement line and the ground line connected to the metal member.

The metal panel crack detection sensor may further include a bonding agent configured to attach the cover to the metal panel.

The power line may not be in contact with the measurement line.

An end of the measurement line may have a pointy shape so that the measurement line is inserted into the cover, and an end of the power line may have a pointy shape so that the power line is inserted into the cover.

The metal panel may include a vehicle metal panel.

The conductive material may include graphite powder.

The metal panel crack detection sensor and the metal panel crack detection device including the same according to the embodiments of the present invention may accurately detect and measure the crack in the metal panel in real time.

### BRIEF DESCRIPTION OF THE DRAWINGS

The brief description of the drawings is provided to more sufficiently understand the drawings used in the detailed description of embodiments of the present invention.

FIG. 1 is a view (longitudinal sectional view) for explaining a metal panel crack detection sensor according to an embodiment of the present invention.

FIG. 2 is a view for explaining a metal panel crack detection device including the metal panel crack detection sensor according to an embodiment of the present invention.

FIG. 3 is a view for explaining an example of an operation of the metal panel crack detection device when a crack is created in a metal panel illustrated in FIG. 2.

The following elements may be used in connection with the drawings to describe embodiments of the present invention.
1: Conductive material
2: Cover
3: Measurement line
4: Power line
5: Bonding agent
12: Insulation coating layer
14: Metal member

### DETAILED DESCRIPTION OF ILLUSTRATIVE EMBODIMENTS

In order to sufficiently understand embodiments of the present invention and the features to be achieved by carrying out the embodiments of the present invention, reference needs to be made to the accompanying drawings for illustrating embodiments of the present invention and the contents disclosed in the accompanying drawings.

Hereinafter, the embodiments of the present invention will be described in detail with reference to the accompanying drawings. In the description of embodiments of the present invention, the specific descriptions of publicly known related configurations or functions will be omitted when it is determined that the specific descriptions may obscure the subject matter of the present invention. Like reference numerals indicated in the respective drawings may refer to like components.

The terms used in the present specification are used only for the purpose of describing particular embodiments and are not intended to limit the present invention. Singular expressions include plural expressions unless clearly described as different meanings in the context. In the with a specification, it should be understood the terms “comprises,” “comprising,” “includes,” “including,” “containing,” “has,” “having” or other variations thereof are inclusive and therefore specify the presence of stated features, integers, steps, operations, elements, components, or combinations thereof, but do not preclude the presence or addition of one or more other features, integers, steps, operations, elements, components, or combinations thereof.

Throughout the present specification, when one constituent element is referred to as being “connected to” another constituent element, one constituent element can be “directly connected to” the other constituent element, and one constituent element can also be “electrically or mechanically connected to” the other constituent element with other constituent elements therebetween.

Unless otherwise defined, the terms used herein, including technical or scientific terms, have the same meaning as commonly understood by those skilled in the art to which the present invention pertains. The terms such as those defined in a commonly used dictionary should be interpreted as having meanings consistent with meanings in the context of related technologies and should not be interpreted as ideal or excessively formal meanings unless explicitly defined in the present specification.

According to the related technologies, in order to evaluate durability of a metal panel, a crack in the metal panel is detected by an operator with the naked eye or by using a penetrant liquid (or a coating liquid).

The method of detecting the crack in the metal panel with the naked eye may decrease accuracy (precision) of detection. The method of detecting the crack in the metal panel using the penetrant liquid may require a long period of detection time.

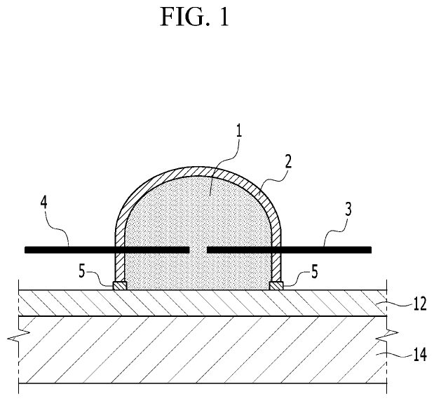

FIG. 1 is a view (longitudinal sectional view) for explaining a metal panel crack detection sensor according to an embodiment of the present invention.

Referring to FIG. 1, the metal panel crack detection sensor may include a conductive material 1 such as graphite powder which is a conductive powder, a cover (or a cap) 2 filled with the conductive material, a measurement line (or a signal line) 3 configured to measure a voltage generated from the conductive material, a power line 4 configured to supply power to the conductive material, and a bonding agent 5 configured to attach the cover to the metal panel.

When the metal panel has a crack, the conductive material 1 (or a part of the conductive material) may move (permeate) into the crack.

For example, the material of the cover 2 may be plastic or rubber.

The metal panel may include a metal member 14, and an insulation coating layer 12 provided on the metal member 14 and including an insulating material. For example, the metal panel may be a vehicle metal panel used for a vehicle body of a vehicle. The vehicle metal panel may include an electrodeposition coating layer formed on the metal member.

The power line 4 may not be in contact with the measurement line 3. For example, an end of the measurement line may have a pointy shape so that the measurement line 3 is inserted into the cover 2. An end of the power line may have a pointy shape so that the power line 4 is inserted into the cover.

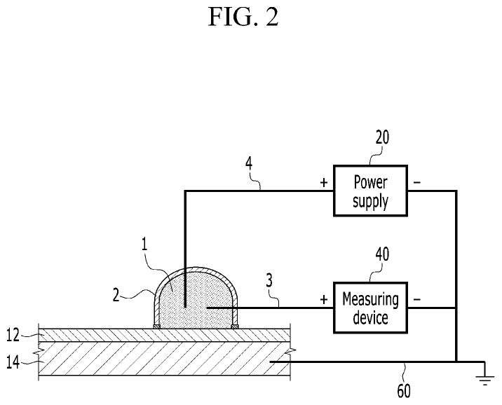

FIG. 2 is a view for explaining a metal panel crack detection device including the metal panel crack detection sensor according to an embodiment of the present invention.

Referring to FIGS. 1 and 2, a metal panel crack detection device (or a metal panel crack detection system) may include the metal panel crack detection sensor illustrated in FIG. 1, a power supply (or a power supply device) 20 configured to supply power to the power line 4 of the metal panel crack detection sensor, a measuring device 40 configured to measure a voltage drop occurring from the measurement line 3 of the metal panel crack detection sensor to the conductive material 1 of the metal panel crack detection sensor, and a ground line 6o connected to a negative terminal of the power supply and a negative terminal of the measuring device.

When the metal panel has a crack, the conductive material 1 moves into the crack, and the measuring device 40 may measure the voltage drop occurring in the conductive material by using the measurement line 3 and the ground line 6o connected to the metal member.

As illustrated in FIG. 2, when the metal panel does not have a crack, no voltage drop occurs in the conductive material 1. Therefore, the measuring device 40 may measure a voltage including no voltage drop. For example, when the power supply 20 supplies a voltage of 5 volts to the conductive material 1, the measuring device 40 may measure the voltage of 5 volts in the conductive material 1.

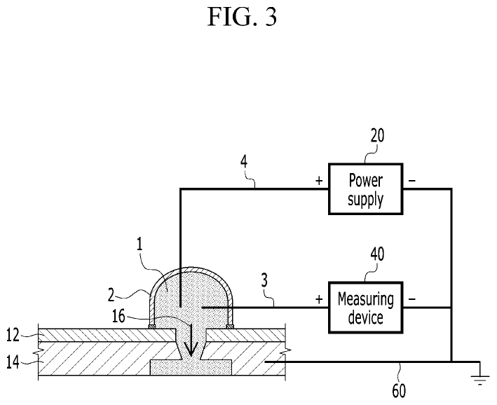

FIG. 3 is a view for explaining an example of an operation of the metal panel crack detection device when a crack is created in the metal panel illustrated in FIG. 2.

As illustrated in FIG. 3, when the metal panel has a crack, a leakage current 16 is generated in the conductive material 1 in the crack, such that a voltage drop may occur in the conductive material 1. Therefore, the measuring device 40 may measure a voltage with the voltage drop. For example, when the power supply 20 supplies a voltage of 5 volts to the conductive material 1, the measuring device 40 may measure a voltage of 40.8 volts in the conductive material by means of the ground line 60 and the measurement line 3.

Based on a voltage drop value, the measuring device 40 may accurately detect the crack in the metal panel and a situation in which the crack grows, thereby evaluating durability of the metal panel.

As described above, the embodiments have been described with reference to the drawings and the specification. In this case, specific terms used herein are used only for the purpose of describing embodiments of the present invention, but are not used to limit the meaning or the scope of the present invention disclosed in the claims. Accordingly, those skilled in the art will understand that various modifications of embodiments of the present invention and other embodiments equivalent thereto may be implemented. Accordingly, the true technical protection scope of the present invention should be determined by the technical spirit of the appended claims.

What is claimed is:

1. A metal panel crack detection sensor comprising:
a cover filled with a conductive material;
a measurement line configured to measure a voltage generated in the conductive material; and
a power line configured to supply power to the conductive material;
wherein the metal panel crack detection sensor is configured to detect whether or not there is a crack in a metal panel that includes a metal member and an insulating coating layer formed on the metal member, the metal panel crack detection sensor being configured to detect a crack in the metal panel when the cover is attached to the metal panel and the conductive material moves into the crack in the metal panel.

2. The metal panel crack detection sensor of claim 1, further comprising a bonding agent configured to attach the cover to the metal panel.

3. The metal panel crack detection sensor of claim 1, wherein the power line is not in physical contact with the measurement line.

4. The metal panel crack detection sensor of claim 1, wherein:
an end of the measurement line has a pointy shape and the end of the measurement line is inserted into the cover; and
an end of the power line has a pointy shape and the end of the power line is inserted into the cover.

5. The metal panel crack detection sensor of claim 1, wherein the metal panel comprises a vehicle metal panel.

6. The metal panel crack detection sensor of claim 1, wherein the conductive material comprises graphite powder.

7. A method of using the metal panel crack detection sensor of claim 1 to detect whether or not there is the crack in the metal panel, the method comprising measuring a voltage drop between the measurement line and the power line.

8. A metal panel crack detection device comprising:
a power supply configured to supply power to a power line of a metal panel crack detection sensor comprising a cover filled with a conductive material, the cover being configured to be attached to a metal panel comprising an insulation coating layer formed on a metal member, wherein the power line is configured to supply power to the conductive material;
a measuring device configured to measure a voltage generated in the conductive material of the metal panel crack detection sensor from a measurement line of the metal panel crack detection sensor, wherein the measurement line is configured to measure a voltage drop occurring in the conductive material; and
a ground line connected to a negative terminal of the power supply and a negative terminal of the measuring device;
wherein the measuring device is configured to measure the voltage drop occurring in the conductive material by using the measurement line and the ground line connected to the metal member when the conductive material moves into a crack in the metal panel.

9. The metal panel crack detection device of claim 8, further comprising a bonding agent configured to attach the cover to the metal panel.

10. The metal panel crack detection device of claim 8, wherein the power line is not in physical contact with the measurement line.

11. The metal panel crack detection device of claim 8, wherein:
an end of the measurement line has a pointy shape and the end of the measurement line is inserted into the cover; and
an end of the power line has a pointy shape and the end of the power line is inserted into the cover.

12. The metal panel crack detection device of claim 11, wherein the power line is not in physical contact with the measurement line.

13. The metal panel crack detection device of claim 8, wherein the metal panel comprises a vehicle metal panel.

14. The metal panel crack detection device of claim 8, wherein the conductive material comprises graphite powder.

15. A method of detecting a crack in a metal panel, the method comprising:
supplying power to a power line of a metal panel crack detection sensor that comprises a cover filled with a conductive material, the cover being attached to the metal panel that comprises an insulation coating layer formed on a metal member, wherein the power line supplies the power to the conductive material;
measuring a voltage generated in the conductive material of the metal panel crack detection sensor from a measurement line of the metal panel crack detection sensor; and
based on the measuring, determining whether a crack exists in the metal panel by detecting a voltage drop in the conductive material, the voltage drop being caused when the conductive material moves into the crack in the metal panel and contacts the metal member.

16. The method of claim 15, further comprising attaching the cover to the metal panel prior to supplying the power.

17. The method of claim 16, wherein the cover is attached to the metal panel using a bonding agent.

18. The method of claim 15, wherein:
an end of the measurement line has a pointy shape and the end of the measurement line is inserted into the cover; and
an end of the power line has a pointy shape and the end of the power line is inserted into the cover.

19. The method of claim 15, wherein the metal panel comprises a vehicle metal panel.

20. The method of claim 15, wherein the conductive material comprises graphite powder.
